(19) United States
(12) Patent Application Publication
(10) Pub. No.: US 2020/0289332 A1
(43) Pub. Date: Sep. 17, 2020

(54) ORTHOPEDIC FIXING MEMBER

(71) Applicant: TOYO ALUMINIUM KABUSHIKI KAISHA, Osaka (JP)

(72) Inventors: Tetsuya MATSUI, Osaka (JP); Hiroki TAMAURA, Osaka (JP); Keisuke IWASAKI, Osaka (JP)

(73) Assignee: TOYO ALUMINIUM KABUSHIKI KAISHA, Osaka (JP)

(21) Appl. No.: 16/776,626

(22) Filed: Jan. 30, 2020

(30) Foreign Application Priority Data
Feb. 1, 2019 (JP) 2019-016984

(51) Int. Cl. A61F 13/04; A61F 5/01; A61F 5/08; A61L 15/08; A61L 15/14

(52) U.S. Cl. CPC A61F 13/04; A61F 5/0118; A61F 5/0111; A61F 2013/51026; A61L 15/08; A61L 15/14; A61F 5/08

(57) ABSTRACT

A fixing member for medical treatment is provided which is constituted by filaments containing a thermoplastic resin and a filler. The filaments are entangled together to form a mesh structure.

### TECHNICAL FIELD

The present invention relates to an orthopedic fixing member.

### BACKGROUND ART

As orthopedic fixing members for fixing affected human body parts, or so-called “casts”, instead of conventional plaster casts, fixing members made of thermoplastic resin material as disclosed in the below-identified Patent Documents 1 and 2 are starting to be widely used nowadays.

Compared to conventional casts such as plaster casts, the orthopedic fixing members of Patent Documents 1 and 2 are advantageous in that, by applying heat, they can be easily deformed along an affected body part, e.g., a fractured body part, and can be easily re-deformed according to a change in condition of the affected body part. Moreover, once hardened, these orthopedic fixing members have sufficient strength.

However, because the orthopedic fixing members of Patent Documents 1 and 2 are plate-shaped members formed from, e.g., a thermoplastic resin, unless they are relatively small in size just enough to fix a finger or a nose, that is, if they are large in size enough to fix a wrist or an ankle, these orthopedic fixing members tend to be so heavy as to give discomfort to the wearer.

Also, since these orthopedic fixing members are plate-shaped and thus relatively fragile, when they are deformed along an affected body part, cracks may form in bent portions thereof where loads concentrate.

Also, since these orthopedic fixing members are plate-shaped, and have smooth surfaces, if two or more of such orthopedic fixing members are superposed on each other, they can easily shift relative to each other such that their interfaces separate from each other.

Moreover, while the orthopedic fixing member of Patent Document 2 is formed with vent holes so as to improve its air permeability, the air permeability is still insufficient, so that heat and moisture tend to be trapped. This may cause the affected body part to become hot and humid, which may in turn cause itchiness or the propagation of germs.

### PRIOR ART DOCUMENTS

### Patent Documents

Patent document 1: Japanese Unexamined Patent Application Publication No. 2018-042721

Patent document 1: Japanese Unexamined Patent Application Publication No. 2018-102492

### SUMMARY OF THE INVENTION

### Problems to be Solved by the Invention

It is an object of the present invention to improve the air permeability of an orthopedic fixing member containing a thermoplastic resin and thus deformable when heated; reduce the likelihood of the formation of cracks in bent portions of the orthopedic fixing member when deformed; reduce the likelihood of the separation of two or more of such fixing members that are superposed on each other; and reduce the weight of the orthopedic fixing member.

### Means for Solving the Problems

In order to achieve the above object, the present invention provides an orthopedic fixing member comprising filaments containing a thermoplastic resin and a filler, the filaments being entangled together to form a mesh structure.

For the orthopedic fixing member of the present invention, the entangled filaments may be fused together at their intersections. Also, the entirety of the orthopedic fixing member may form a sheet shape. Also, the filler may be a heat-conducting powder higher in thermal conductivity than the thermoplastic resin, and is preferably a flake-shaped aluminum powder. The filler contained in the filaments may be present in an amount of 5 parts by mass or more and 50 parts by mass or less, relative to 100 parts by mass of the thermoplastic resin.

### Effects of the Invention

Since the orthopedic fixing member of the present invention has a mesh structure, and thus has gaps larger than those of conventional plate-shaped orthopedic fixing members, the orthopedic fixing member of the present invention has improved air permeability, and is light in weight.

Also, since the orthopedic fixing member of the present invention contains a filler in addition to a thermoplastic resin, it has improved strength compared to fixing members containing only a thermoplastic resin.

In particular, when deformed along an affected body part, the mesh structure is partially compressed and partially stretched in such a manner as to improve the bending strength at bent portions to which large loads tend to be applied, thereby reducing the likelihood of the formation of cracks in and from the bent portions.

Also, when two or more of the orthopedic fixing members according to the present invention are superposed on each other, since heated and softened filaments are entangled together on the superposed surfaces of the orthopedic fixing members, the superposed surfaces are less likely to separate from each other. Therefore, by superposing two or more of them as necessary, i.e., according to the position and size of the affected body part, it is possible to partially reinforce the affected body part easily.

### BRIEF DESCRIPTION OF THE DRAWINGS

FIG. 1A is a plan view of an orthopedic fixing member embodying the present invention; and FIG. 1B is a side view thereof.

FIGS. 2A to 2D are perspective views showing, as an example, how the orthopedic fixing member of the present invention is used.

FIG. 3A is a side view showing, as a different example, how the orthopedic fixing member of the present invention is used.

FIG. 3B is a perspective view showing, as a still different example, how the orthopedic fixing member of the present invention is used.

FIG. 3C is a side view showing, as a yet different example, how the orthopedic fixing member of the present invention is used.

FIG. 4 is a side view of the orthopedic fixing member of the present invention when deformed by bending.

### BEST MODE FOR CARRYING OUT THE INVENTION

The embodiment of the present invention is now described with reference to the drawings.

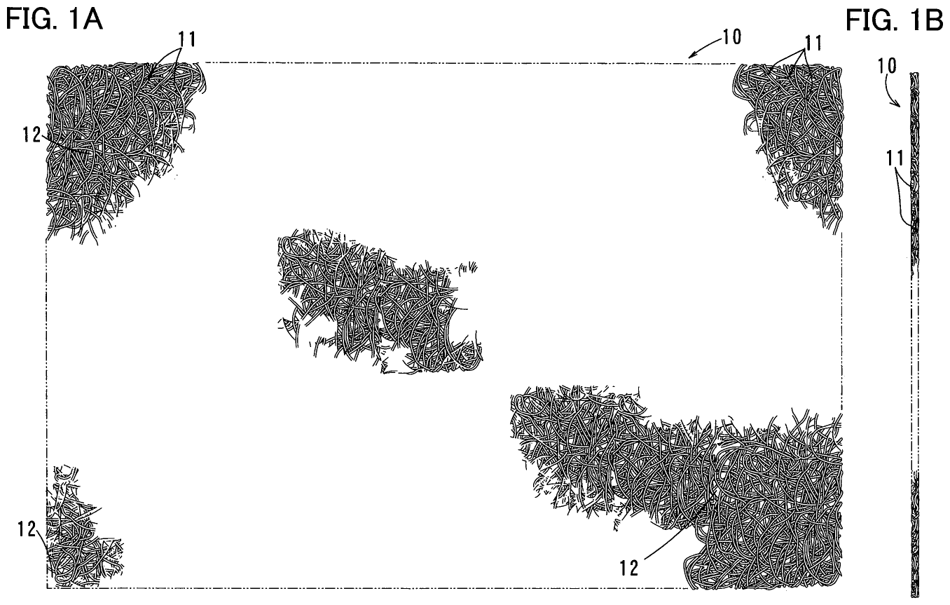

As illustrated in FIGS. 1A and 1B, the orthopedic fixing member 10 of the embodiment is a substantially rectangular (in plan view) sheet-shaped member formed by entangling filaments 11 containing a thermoplastic resin and a filler.

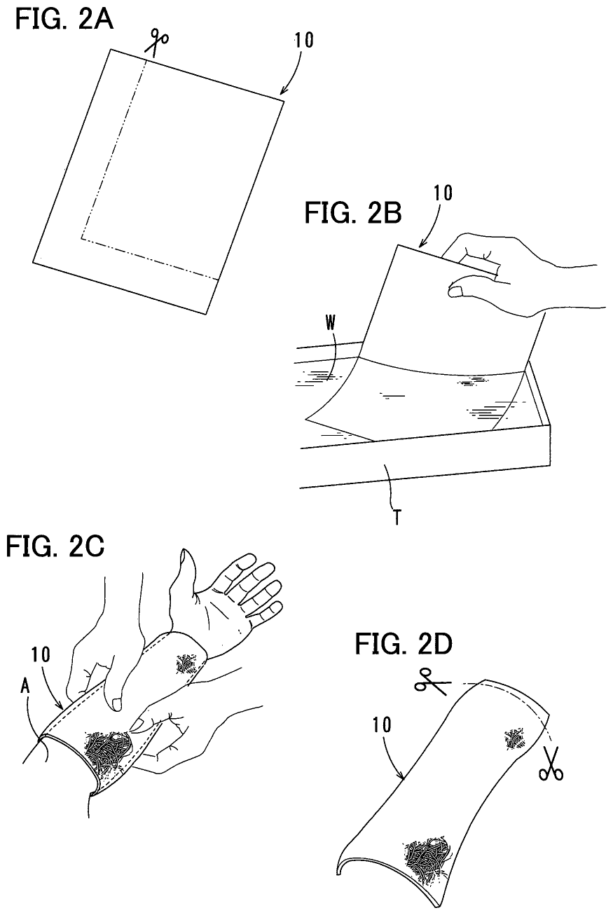

To use this sheet-shaped orthopedic fixing member 10, as illustrated in FIG. 2A, the sheet-shaped member is first appropriately cut according to the size of an affected human body part. The sheet-shaped member is then heated and softened, e.g., by soaking it in hot water W in a tray T as illustrated in FIG. 2B. Then, after deforming the softened sheet-shaped member along the affected body part (arm A in the shown example) as illustrated in FIG. 2C, it is hardened, e.g., by naturally cooling it, thereby fixing the affected body part A.

When the condition of the affected body part A changes, e.g., when the swelling of the affected body part A becomes small, the orthopedic fixing member is removed from the affected body part A, and adjusted to fit the altered affected body part A, by re-deforming it by heating, and then e.g., by appropriately cutting any portion of the orthopedic fixing member which is no longer necessary as illustrated in FIG. 2D.

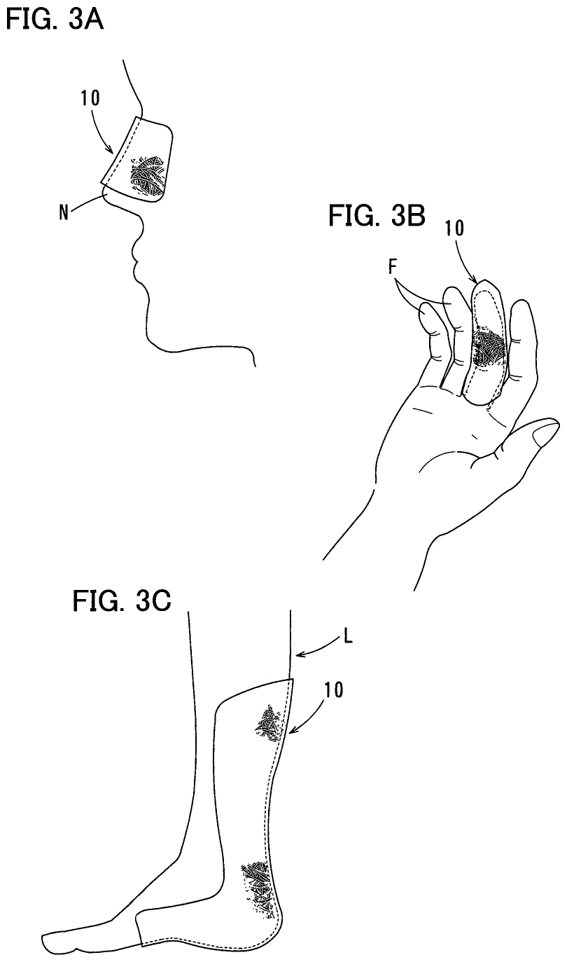

While the affected body part is exemplified as an arm A in FIGS. 2A to 2D, the orthopedic fixing member 10 of the present invention can be used for other affected body parts, such as a nose N as illustrated in FIG. 3A, a finger F as illustrated in FIG. 3B, or a leg L as illustrated in FIG. 3C. In such cases, too, in the same manner as described above, the orthopedic fixing member is cut, softened and deformed along an affected body part, and hardened by cooling, thereby fixing the affected body part.

Since the orthopedic fixing member 10 of the present invention is a sheet-shaped member as illustrated in FIGS. 1A and 1B, it can be easily handled, for example, it can be easily deformed along an affected body part.

The dimensions of the sheet-shaped member are not particularly limited, but the length and width dimensions thereof are preferably, e.g., 300 to 500 mm. This is because, if the length and width dimensions are too large, e.g., several thousand millimeters, it will be difficult to handle such a large sheet-shaped member, whereas, if the length and width dimensions are too small, e.g., several tens of millimeters, it will be difficult to fully cover a relatively large affected body part with such a small sheet-shaped member.

On the other hand, the thickness dimension of the sheet-shaped member is preferably, e.g., 3 to 10 mm. This is because, if the thickness dimension is less than 3 mm, the strength of the sheet-shaped member will decrease, and also cracks may form in bent portions of the sheet-shaped member when it is deformed, whereas, if the thickness dimension is less than 10 mm, it may be difficult to deform the sheet-shaped member, and also the weight of the sheet-shaped member will increase, which could give discomfort to the wearer.

The filaments 11, which form the orthopedic fixing member 10, are overlapped and entangled together while being arranged substantially in circular arcs in plan view as illustrated in FIG. 1A, and also entangled together in the thickness direction of the orthopedic fixing member 10 as illustrated in FIG. 1B, thereby constituting a three-dimensional mesh structure. The intersection points (contact points) of the entangled filaments 11 are fused together.

The three-dimensional mesh structure defines gaps 12 penetrating through the orthopedic fixing member 10 in its thickness direction, so that the orthopedic fixing member 10 has air permeability in its thickness direction.

Due to such a three-dimensional mesh structure constituted by the filaments 11, the gaps 12 of the orthopedic fixing member 10 of the present invention are larger than those of conventional plate-shaped orthopedic fixing members. Therefore, the orthopedic fixing member 10 is light in weight, and excellent in air permeability. The value of the air permeability of the orthopedic fixing member 10 is not particularly limited, and may be, e.g., 150 to 250 cm³/cm²·s as measured according to the JIS L 1096 A method.

The density of the three-dimensional mesh structure constituted by the filaments 11 is not particularly limited, but is preferably 0.1 to 1 g/cm³. This is because, if the density is less than 0.1 g/cm³, the strength of the orthopedic fixing member 10 may decrease, whereas, if the density is more than 1 g/cm³, the air permeability of the orthopedic fixing member 10 may deteriorate. For the same reason, the porosity of the orthopedic fixing member 10 is preferably 4 to 90%.

Due to the three-dimensional mesh structure constituted by the filaments 11, if two or more of the orthopedic fixing members 10 according to the present invention need to be superposed on each other, e.g., for reinforcement, since heated and softened filaments are hardened while entangled together on the superposed surfaces (interfaces) of the orthopedic fixing members 10, the interfaces of the orthopedic fixing members 10 are less likely to separate from each other compared to conventional orthopedic fixing members having smooth outer surfaces.

Since the intersection points of the filaments 11 are fused together, the filaments are prevented from separating from each other. Also, since the filaments 11 contain a filler in addition to a thermoplastic resin, they are higher in strength than filaments composed only of a thermoplastic resin.

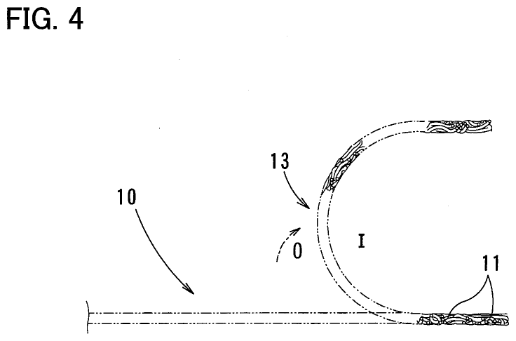

When the orthopedic fixing member 10 is deformed by bending as illustrated in FIG. 4, the mesh structure is compressed on the inner side I of the bent portion 13 of the orthopedic fixing member 10, and stretched on the outer side O of the bent portion 13. This absorbs displacement due to bending, and thus prevents cracks in or from the bent portion 13.

The value of the flexural strength of the orthopedic fixing member 10 is not particularly limited, and may be, e.g., 65 to 85 N.

The thickness, sectional shape and length of the filaments are not particularly limited, provided that the filaments have a fiber-like shape, and can form a three-dimensional mesh structure by fusing their intersection points together. However, the thickness may be 0.1 to 5 mm so that the filaments have a sufficient strength, and the sectional shape may be, e.g., a circular, elliptical or polygonal shape.

Also, the filaments, which contain a thermoplastic resin and a filler as essential components, may further contain other optionally selected components such as an antibacterial agent, a fungicide and an agent for giving a cold feeling to the touch.

The thermoplastic resin contained in the filaments is not particularly limited in kind, and may be, e.g., a polyester resin such as polycaprolactone (PCL), polylactic acid, or polyglycolic acid; or a polyolefin resin such as polyethylene (PE) or polypropylene (PP). Also, a mixture of these resins may be used instead.

The melting temperature range of the thermoplastic resin is not particularly limited, but is preferably 40 degrees Celsius or more and 90 degrees Celsius or less, because, within this melting temperature range, it is possible to easily soften the orthopedic fixing member in hot water of a generally used temperature. Polycaprolactone is particularly preferably used as the thermoplastic resin, because, having a melting temperature range of 58 to 60 degrees Celsius, polycaprolactone is easily thermally deformed in hot water of about 50 to 80 degrees Celsius, and also, once hardened, polycaprolactone is not easily deformable.

If polycaprolactone is used as the thermoplastic resin, it is suitable to use any of the thermoplastic polycaprolactones produced by Perstorp carrying the following grade names: “Capa™6100”, “Capa™6200”, “Capa™6250”, “Capa™6400”, “Capa™6430”, “Capa™6500”, “Capa™6500C”, “Capa™6506”, and “Capa™6800”. These polycaprolactones are preferable because they allow the orthopedic fixing member to be easily deformed along an affected body part, simply by soaking it in hot water of about 60 degrees Celsius or more (optimally in hot water of 90 degrees Celsius for about 3 seconds).

The filler contained in the filaments is not particularly limited in kind, provided that the filler can improve the strength of the orthopedic fixing member when mixed with the thermoplastic resin, but the filler is preferably light in weight so as not to increase the weight of the orthopedic fixing member.

Also, a substance having a high thermal conductivity is preferably used as the filler, because by using such a filler, it is possible to effectively release the heat of the affected body part through the orthopedic fixing member to the outside.

Such substances include, for example, metals such as aluminum, magnesium and iron; their oxides and metallic compounds; their alloys; and inorganic substances such as silica. Among them, an aluminum powder (including an aluminum alloy powder) is especially preferable because an aluminum powder is inexpensive, easily available, light in weight, and has a high thermal conductivity. The particle diameter of an aluminum powder used is not particularly limited. For example, an aluminum powder having an average particle diameter of 5 to 10 μm may be used.

The amount of the filler is preferably 10 parts by mass or more and 50 parts by mass or less, more preferably 10 parts by mass or more and 20 parts by mass or less, relative to 100 parts by mass of the thermoplastic resin. This is because, if this amount is less than 10 parts by mass, the filler may be unable to sufficiently improve the strength of the orthopedic fixing member, whereas, if the amount is more than 20 parts by mass, the filler may increase the total weight of the orthopedic fixing member.

The shape of the filler is not particularly limited, and may be, e.g., a granular shape, a substantially spherical shape, a flake shape, or an irregular shape. The filler may be subjected to a surface treatment.

The method for manufacturing the orthopedic fixing member of the present invention is not particularly limited. For example, the orthopedic fixing member may be manufactured by a method for manufacturing a nonwoven fabric, i.e., by spinning, in the form of filaments, a mixture of a molten thermoplastic resin and a filler from nozzles, and piling the spun filaments on a collecting conveyor.

By appropriately adjusting the movement of the nozzles and the shape and position of the spinning outlets of the nozzles, the filaments composed of the above mixture are piled on the collecting conveyor in circular arcs, and their intersection points are fused together by their own weights before being naturally cooled and hardened. Since the filaments are in point contact with each other, and loads concentrate on these contact points, the filaments are firmly fused together.

The contents of the present invention are further clarified by the below-described Examples and Comparative Examples.

### EXAMPLE 1

First, a composition was prepared by kneading together: 80 parts by mass of a polycaprolactone resin (product name Capa™6800, produced by Perstorp) as the thermoplastic resin; and 20 parts by mass of a masterbatch containing aluminum flakes (product name: “METAX NEO”; product number: “NME010T6”; aluminum content: 70% by weight; aluminum powder average particle diameter: 10 μm; carrier resin: mixture of low density polyethylene and polyethylene wax; produced by Toyo Aluminium K.K.).

Then, using an extruder having nozzles, the thus-obtained composition was extruded at 200 degrees Celsius, at an extrusion speed of 18 cm/min, and in the form of molten thread-shaped filaments measuring about 1.3 mm.

When extruding the composition, by entangling the thread-shaped filaments together before being hardened, a rectangular sheet-shaped orthopedic fixing member according to Example 1 was prepared which measures 450 mm in length×300 mm in width×5 mm in thickness, and which has a three-dimensional mesh structure.

### EXAMPLE 2

An orthopedic fixing member according to Example 2 was prepared in the same manner as Example 1 except that the mixture ratio of the polycaprolactone resin to the masterbatch containing aluminum flakes was 60 parts by mass to 40 parts by mass.

### COMPARATIVE EXAMPLE 1

A composition was prepared by kneading together: 60 parts by mass of a polycaprolactone resin (product name Capa™6800, produced by Perstorp) as the thermoplastic resin; and 40 parts by mass of a masterbatch containing aluminum flakes (product name: “METAX NEO”; product number: “NME010T6”; aluminum content: 70% by weight; aluminum powder average particle diameter: 10 μm; carrier resin: mixture of low density polyethylene and polyethylene wax; produced by Toyo Aluminium K.K.). Then, using a general purpose injection molder, the thus-obtained composition was formed into a plate-shaped product measuring 100 mm in length×100 mm in width×2 mm in thickness.

Then, the plate-shaped product was cut, corresponding to the dimensions of the orthopedic fixing member of Example 1, to 450 mm in length×300 mm in width, to provide an orthopedic fixing member according to Comparative Example 1.

### COMPARATIVE EXAMPLE 2

An orthopedic fixing member according to Comparative Example 2 was prepared in the same manner as Example 1 except that only the polycaprolactone resin was used as the raw material.

### Test 1

The weights of the orthopedic fixing members of Example 1 and Comparative Example 1 were measured.

The measurement results are shown in Table 1. As can be seen from Table 1, the orthopedic fixing member of Example 1 is lighter in weight by about 5% than the orthopedic fixing member of Comparative Example 1.

TABLE 1

| | Example 1 | Comparative Example 1 |
|---|---|---|
| Weight (g) | 320 | 338 |

### Test 2

The air permeabilities of the orthopedic fixing members of Examples 1 and 2 and Comparative Example 1 were measured by the JIS L 1096 A method.

The measurement results are shown in Table 2. As can be seen from Table 2, the air permeabilities of the orthopedic fixing members of Examples 1 and 2 drastically improved compared to that of the orthopedic fixing member of Comparative Example 1.

TABLE 2

| | | | Comparative |
|---|---|---|---|
| | Example 1 | Example 2 | Example 1 |
| Air permeability | 210 | 194 | 40 |
| (cm³/cm² - s) | | | |

### Test 3

Test pieces each having a length of 100 mm and a width of 100 mm were cut out of the respective orthopedic fixing members of Examples 1 and 2 and Comparative Examples 1 and 2, and their flexural strengths were measured by folding the test pieces at their ends.

The flexural strengths were measured using a 5969 type universal testing machine (manufactured by Instron). Specifically, with each test piece set on the table of the testing machine, which is initially spaced 8 cm from the head of the testing machine, the head was lowered toward the table at 10 mm/min, and the load when the test piece was broken was measured. This measurement was performed five times, and the average of the five measured values was calculated as the flexural strength.

The measurement results are shown in Table 3. As can be seen from Table 3, the flexural strengths of the orthopedic fixing members of Examples 1 and 2 are large enough to be comparable to that of the orthopedic fixing member of Comparative Example 1, and are significantly larger than that of the orthopedic fixing member of Comparative Example 2.

TABLE 3

| | | | Comparative | Comparative |
|---|---|---|---|---|
| | Example 1 | Example 2 | Example 1 | Example 2 |
| Flexural | 77 | 78 | 81 | 57 |
| strength (N) | | | | |

The above-described embodiment and Examples are for illustrative purposes only in every respect, and the present invention is not limited thereto. The scope of the present invention is indicated by the claims, and covers all modifications and variations within the scope of the claims and the meaning equivalent thereto.

For example, while the orthopedic fixing member 10 of the embodiment is a sheet-shaped member, the orthopedic fixing member of the present invention is not limited thereto, and may be irregular-shaped. On the other hand, if the orthopedic fixing member 10 is sheet-shaped, its shape in plan view is not limited to a rectangular shape as shown in the embodiment, and may be, for example, a square shape, a triangular shape, a circular shape or an elliptical shape, provided that the orthopedic fixing member 10 can fit the shape of the affected body part.

While the filaments 11 of the orthopedic fixing member 10 are arranged in circular arcs in plan view in the embodiment, they may be arranged differently, for example, substantially in straight lines, or in wavy or irregular-shaped patterns, in plan view.

While the orthopedic fixing member 10 is constituted by only the filaments 11 in the embodiment, it may include an additional element or elements. For example, a cooling sheet for cooling the affected body part may be laminated on one surface of the three-dimensional mesh structure constituted by the filaments 11.

While the intersection points of the filaments 11 are fused together in the embodiment, they may be fixed to each other by another means, e.g., fixedly bonded together with an adhesive, or may be simply superposed on each other without being fixed to each other.

### DESCRIPTION OF REFERENCE NUMERALS

10: Orthopedic fixing member
11: Filament
12: Gap
13: Bent portion
A: Arm
N: Nose
F: Finger
L: Leg
T: Tray
W: Hot water
I: Inner side of the bent portion
O: Outer side of the bent portion

1. An orthopedic fixing member comprising filaments containing a thermoplastic resin and a filler,
wherein the filaments are entangled together to form a mesh structure.

2. The orthopedic fixing member according to claim 1, wherein the entangled filaments are fused together at intersections of the filaments.

3. The orthopedic fixing member according to claim 1, wherein an entirety of the orthopedic fixing member forms a sheet shape.

4. The orthopedic fixing member according to claim 1, wherein the filler is a heat-conducting powder higher in thermal conductivity than the thermoplastic resin.

5. The orthopedic fixing member according to claim 4, wherein the heat-conducting powder is a flake-shaped aluminum powder.

6. The orthopedic fixing member according to claim 1, wherein the filler contained in the filaments is present in an amount of 5 parts by mass or more and 50 parts by mass or less, relative to 100 parts by mass of the thermoplastic resin.

7. The orthopedic fixing member according to claim 1, wherein the entangled filaments are fused together at intersections of the filaments, and an entirety of the orthopedic fixing member forms a sheet shape.

8. The orthopedic fixing member according to claim 7, wherein the filler is a heat-conducting powder higher in thermal conductivity than the thermoplastic resin.

9. The orthopedic fixing member according to claim 8, wherein the heat-conducting powder is a flake-shaped aluminum powder.

10. The orthopedic fixing member according to claim 7, wherein the filler contained in the filaments is present in an amount of 5 parts by mass or more and 50 parts by mass or less, relative to 100 parts by mass of the thermoplastic resin.

11. The orthopedic fixing member according to claim 1, wherein the entangled filaments are fused together at intersections of the filaments, and the filler is a heat-conducting powder higher in thermal conductivity than the thermoplastic resin.

12. The orthopedic fixing member according to claim 11, wherein the heat-conducting powder is a flake-shaped aluminum powder.

13. The orthopedic fixing member according to claim 11, wherein the filler included in the filaments is present in an amount of 5 parts by mass or more and 50 parts by mass or less, relative to 100 parts by mass of the thermoplastic resin.

14. The orthopedic fixing member according to claim 1, wherein an entirety of the orthopedic fixing member forms a sheet shape, and the filler is a heat-conducting powder higher in thermal conductivity than the thermoplastic resin.

15. The orthopedic fixing member according to claim 14, wherein the heat-conducting powder is a flake-shaped aluminum powder.

16. The orthopedic fixing member according to claim 14, wherein the filler contained in the filaments is present in an amount of 5 parts by mass or more and 50 parts by mass or less, relative to 100 parts by mass of the thermoplastic resin.

17. The orthopedic fixing member according to claim 1, wherein the entangled filaments are fused together at intersections of the filaments,
wherein an entirety of the orthopedic fixing member forms a sheet shape,
wherein the filler is a heat-conducting powder higher in thermal conductivity than the thermoplastic resin, and
wherein the filler contained in the filaments is present in an amount of 5 parts by mass or more and 50 parts by mass or less, relative to 100 parts by mass of the thermoplastic resin.

18. The orthopedic fixing member according to claim 17, wherein the heat-conducting powder is a flake-shaped aluminum powder.
